(19) United States
(12) Patent Application Publication
(10) Pub. No.: US 2022/0220030 A1
(43) Pub. Date: Jul. 14, 2022

(54) GLASS COMPOSITE MATERIAL AND METHOD FOR PRODUCING

(71) Applicant: SCHOTT AG, Mainz (DE)

(72) Inventor: Oliver KIRCHNER, Jena (DE)

(73) Assignee: SCHOTT AG, Mainz (DE)

(21) Appl. No.: 17/708,456

(22) Filed: Mar. 30, 2022

Related U.S. Application Data
(63) Continuation of application No. PCT/EP2020/077116, filed on Sep. 28, 2020.

(30) Foreign Application Priority Data
Sep. 30, 2019 (DE) 10 2019 215 075.6

(51) Int. Cl. C03C 27/10; B32B 17/06; B32B 3/30; B32B 3/26; B32B 7/12; C03C 3/085; C03C 3/091; C03C 3/093; B01L 3/00

(52) U.S. Cl. CPC C03C 27/10; B32B 17/06; B32B 3/30; B32B 3/266; B01L 2300/0887; C03C 3/085; C03C 3/091; C03C 3/093; B01L 3/502707; B32B 7/12

(57) ABSTRACT

A glass composite is provided that has a first and second glass element, each having a first surface, and a first coupling agent layer having a first and second silane coupling agent. The first coupling agent layer has covalent bonds between the first and second silane coupling agents. The first and second silane coupling agents are covalently bonded to the first surface of the first and second glass elements, respectively. The first and second glass elements are irreversibly connected by the first coupling agent layer. Such a glass composite is made by bonding the first surface of the first and second glass elements to the first and second silane coupling agents, respectively, and contacting both first surfaces with each other to cause the first and second silane coupling agents thereon to covalently bond so that the first and second glass elements are irreversibly connected.

### CROSS-REFERENCE TO RELATED APPLICATIONS

This application is a continuation of International Application PCT/EP2020/077116, filed Sep. 28, 2020, which claims priority to German patent application 102019215075.6, filed Sep. 30, 2019, the entire contents of each of which is incorporated by reference herein.

### BACKGROUND

### 1. Field of the Disclosure

The present disclosure relates to a glass composite material, a device comprising the glass composite material and a method for producing the same. The present disclosure also relates to a method of analysing biological samples with the device.

### 2. Description of Related Art

From practice, various composite materials are known, which comprise various glass elements.

Conventional methods for producing such composites include any one of the known joining methods, including gluing, laser welding, contact bonding, thermal or chemical bonding etc.

A method for joining glass elements in the manufacture of a composite material with the aid of an adhesive is described, for example, in DE 102018209589 A1. The use of adhesives in the production of composite materials is problematic, as this often leads to the occurrence of unavoidable, unfavourable and/or unpredictable variations in the thickness of the adhesive layer and thus in the thickness variation of the entire composite. Further, when connecting an element, wetted with adhesive, with a corresponding counterpart element as well as when curing the adhesive, unfavourable tensions between the connected elements in the composite material can remain in the composite material. The use of adhesives for joining glass elements, which find use in biotechnological analytics, holds further risks. For example, through the thickness variation of the adhesive layer, a glass composite material, which is used in the formation of micro-fluidic channels, can lead to an unacceptable variance in the volume of the channel, and can thereby lead to incorrect determination of results. Furthermore, it can be difficult to remove residual amounts of adhesive from the glasses to be joined, which can lead to contamination of a sample. Furthermore, composite materials in biotechnological procedures are regularly used over longer periods of time, for example, are used for several days with aggressive dyeing and/or buffer solutions and are exposed to high temperatures and rapid temperature differences, which result in unfavourable outgassing or bleeding of components of the adhesive, which lead to incorrect determination of results, in particular in fluorescence-based analysis methods.

WO2017/035770A1 describes the joining of an ultra-thin glass to a carrier element by contact bonding, wherein the ultra-thin glass and the substrate remain interconnected via electrostatic forces alone. However, in this method, the two components or elements are not irreversibly connected to each other.

A method of chemical joining thin glass with a glass substrate is described in WO2019/100050. In this method, a temporary joint connection between the two glass elements is sought in order to facilitate the processing of the thin glass even at temperatures of up to 500° C. As the described composite arrangement is reversed after machining the thin glass, it is not irreversible.

Furthermore, various joining methods, such as laser welding, are not suitable for connecting planar elements over their entire surface. Laser welding usually allows connection by a welding seam or by multiple welding points.

Connecting or joining coated glasses, as are regularly used in biotechnological analytics, further limits the possible joining methods. The reasons for this are that the coating masks relevant surface characteristics of the glass elements, which are needed to form a joining connection as required in low temperature wafer bonding, that the coating is incompatible with the adhesive to be used, or that the coating is damaged or unusable by the joining method (thermal bonding).

### SUMMARY

It is therefore an object of the present disclosure to develop a glass composite material of the type mentioned above in such a way that an irreversible joint is provided between the glass elements of the glass composite material, as well as a method for producing the glass composite material. Another object of the present disclosure is to provide both a device, in particular a device for use in biotechnological analysis methods, and a method for its preparation, as well as its use for the analysis of biological samples.

In one embodiment, the present disclosure solves the above-mentioned objects with a glass composite material comprising at least one first glass element, a coupling agent layer and a second glass element, wherein a plurality of first silane coupling agents are covalently bonded to a first surface of the first glass element, and wherein a variety of second silane coupling agents are covalently bonded to a first surface of the second glass element, characterized in that the coupling agent layer is formed by covalent bonds between the first and second silane coupling agents, so that the first glass element is irreversibly connected with the second glass element through the coupling agent layer.

An irreversible connection, is understood to be a connection, which can be permanent and cannot be severed non-destructively, e.g. which cannot be severed without breaking a glass element or without damaging the coupling agent. After severing an irreversible connection, it is not possible to restore the connection. In contrast, reversible connections can be severed non-destructively and can be reconnected, often several times.

Glass elements covalently coated with reactive amino, epoxy or aldehyde silanes, reactive 3-D hydrogels or 3-D polymers are known and are regularly used in biotechnological analytics.

In one manner according to the present disclosure, it has been first recognized that, in a surprisingly simple manner, a connection between at least two complementarily coated glass elements can be realized, by covalently bonding complementarily reactive groups of silane coupling agents to each other, such that they irreversibly connect the coated glass elements (namely over the entire, corresponding surfaces coated with silane coupling agents coated surfaces) with each other. Furthermore, according to the present disclosure, the glass composite material can comprise further glass elements, which—in turn—can again be joined via complementarily reactive silane coupling agents. In particular, passageways in the composite material can be generated through arrangement or surface structuring of the glass elements in accordance with the present disclosure, wherein the inner surfaces of the passageways comprise the respective reactive and functional glass coatings. Therefore, in accordance with the present disclosure, the use of a device comprising the glass composite material according to the present disclosure is also provided, in particular the use in the analysis of biological samples is particularly advantageous as the individual glass elements already comprise particularly suitable coatings for such analysis.

“Glass elements” in the sense of this disclosure, i.e. glass elements used in the present disclosure described herein, are expressly understood to be macroscopic glass elements. Thus, the term “glass element” here expressly excludes microscopic glass particles, in particular nanoparticles, as used in glass powders for coating of a glass surface. Instead, the glass elements of this disclosure are glass components having at least one surface, which can be associated with a corresponding surface of another glass element. Preferably, glass elements in the sense of this disclosure are designed flat, in particular as planar elements (e.g., glass panes), which can stackably be associated with each other via their respective corresponding surfaces.

According to an advantageous embodiment, the glass composite material according to the present disclosure can comprise a third glass element and a further coupling agent layer, wherein: a plurality of the first silane coupling agents is covalently bonded to a second surface of the second glass element, and wherein a plurality of the second silane coupling agents is covalently bonded to a first surface of the third glass element, such that the further coupling agent layer is formed through covalent bonds between the first and second silane coupling agents, so that the second is irreversibly connected to the third glass element by the further coupling agent layer; or a plurality of the second silane coupling agents is covalently bonded to a second surface of the second glass element, and wherein a plurality of the first silane coupling agents is covalently bonded to a first surface of the third glass element such that the further coupling agent layer is formed through covalent bonds between the first and second silane coupling agents, so that the second is irreversibly connected to the third glass element by the further coupling agent layer.

In the following, some of the advantageous embodiments of the glass composite material according to the present disclosure, which are realised through combination of complementarily reactive first and second silane coupling agents, are described. Preferably: when the first silane coupling agent is selected from silane coupling agents and combinations of silane coupling agents, which comprise reactive epoxy, aldehyde or polymer groups, the second silane coupling agent is a reactive amino group; or when the second silane coupling agent is selected from silane coupling agents and combinations of silane coupling agents, which comprise reactive epoxy, aldehyde and polymer groups, the first silane coupling agent is a reactive amino group; or when the first silane coupling agent is selected from silane coupling agents and combinations of silane coupling agents, which comprise reactive epoxy groups, the second silane coupling agent is a reactive thiol group; or when the second silane coupling agent is selected from silane coupling agents and combinations of silane coupling agents, which comprise reactive epoxy groups, the first silane coupling agent is a reactive thiol group.

In the context of this disclosure, the term “silane coupling agent” encompasses silanes with reactive epoxy, aldehyde, thiol, amino or polymer groups, which are covalently bonded to a glass surface.

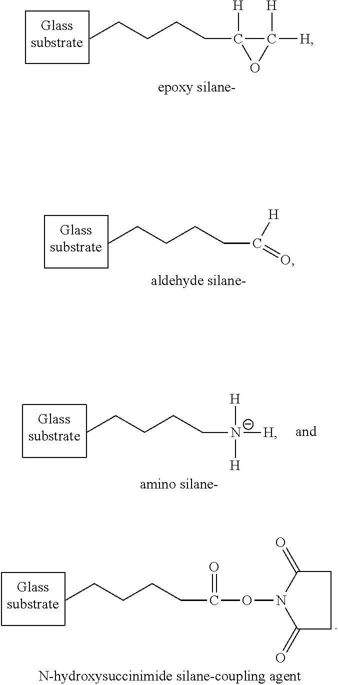

In particular, N-hydroxysuccinimide silane coupling agents can include a polymer in addition to the reactive ester group of the N-hydroxysuccinimide, so that crosslinking between the reactive N-hydroxysuccinimide silane coupling agents is possible. In advantageous embodiments of the present disclosure, a glass element can be coated with a cross-linked “polymer silane coupling agent”.

Amino silane, epoxy silane and/or N-hydroxysuccinimide silane coupling agent coatings are particularly suitable to bind or immobilize oligonucleotide molecules. Furthermore, epoxy silane and/or aldehyde silane coupling agent coatings are particularly suitable for binding peptides, and aldehyde silane, epoxy silane and/or N-hydroxysuccinimide silane coupling agent coatings are particularly suitable for binding proteins.

In the context of this disclosure, the terms “first” and “second” coupling agents are to be understood in their broadest sense. In particular, they neither indicate temporal order or a preference with regard to their selection. Instead, the terms only indicate that the covalent connection is conveyed by two complementarily reactive silane coupling agents, namely between a “first” and a “second” silane coupling agent.

In this case, “complementarily reactive” silane coupling agents are such silane coupling agents, which can engage with each other in a covalent bonding reaction. For example, amino silane coupling agents are complementarily reactive with epoxy silane, aldehyde silane and polymer silane coupling agents.

Preferably, an epoxy silane coupling agent can form a covalent bond with an amino silane or thio silane coupling agent.

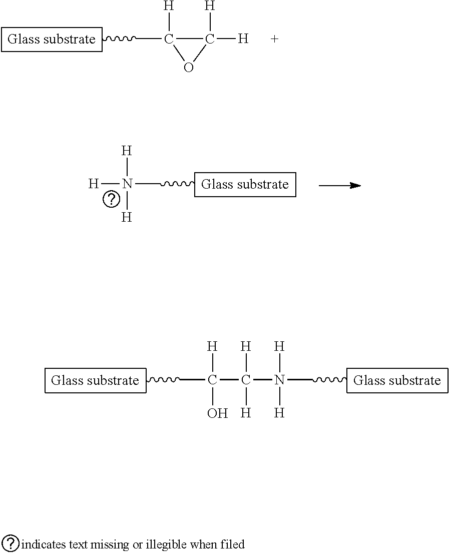

In a particularly advantageous manner, in the bonding reaction between an epoxy silane coupling agent and an amino silane or a thio silane coupling agent no condensation product is generated, which remains in the coupling agent layer. Preferably, an aldehyde silane coupling agent can form a covalent bond with an amino silane coupling agent:

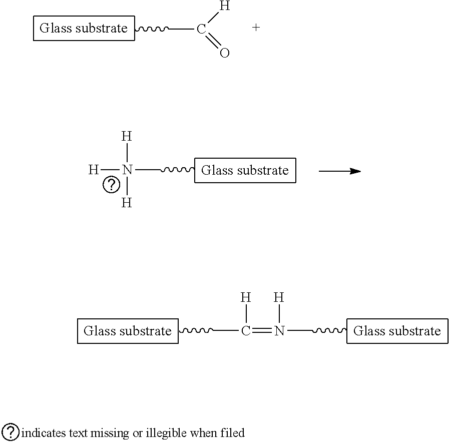

Preferably, a polymer silane coupling agent can form a covalent bond with an amino silane coupling agent:

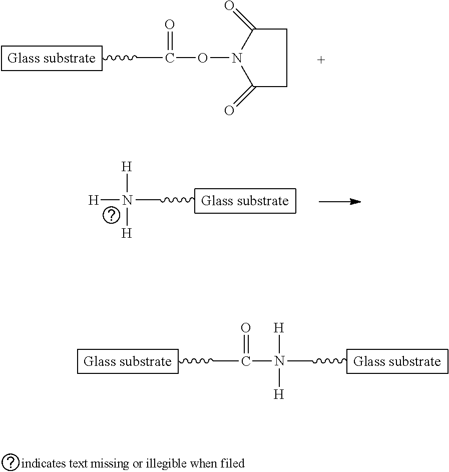

Advantageously, the corresponding surfaces of the glass elements to be connected with each other via the silane coupling agents have a roughness and/or surface structure, which ensures a distance between the surfaces that allows the covalent bond between the respective silane coupling agents. In particularly advantageous embodiments, the connection of the glass elements is made possible, without the forming of cavities in the coupling agent layer, which compromise the structural integrity of the glass composite material.

With respect to the binding reactivity of the silane coupling agents, this distance can also be referred to as the effective distance. In this context, it is understood that corresponding surfaces are those surfaces of the glass elements, which are connected to each other in the glass composite material via the coupling agent layer, and which correspond in their respective surface structure such that they can be brought into relative effective distance to each other. If the distance between two surfaces coated with complementarily reactive silane coupling agents is smaller or equal to the effective distance, a bonding reaction occurs, so that the complementarily reactive silane coupling agents form a covalent bond and form the coupling agent layer of the glass composite material according to the present disclosure and, thereby, covalently and irreversibly connect the surfaces of the glass elements, or the glass elements, with each other. In contrast to glue layers known in the art, the so generated coupling agent layer forms a particularly thin and homogeneous layer with a thickness variation to be neglected. In advantageous embodiments of the present disclosure, the thickness of the coupling agent layer is less than 20 nm, preferably less than 10 nm, and more preferably less than 5 nm.

With regard to the glass elements to be used, it is conceivable that these are selected from: soda-lime glass elements, borosilicate glass elements, quartz glass elements and/or alkaline alumino borosilicate glass elements.

Preferably, the glass of a glass element to be used in the glass composite material according to the present disclosure can have the following composition according to a lithium-aluminum silicate glass (in weight %):

| | |
|---|---|
| SiO₂ | 55-69 |
| Al₂O₃ | 18-25 |
| Li₂O | 3-5 |
| Na₂O + K₂O | 0-30 |
| MgO + CaO + SrO + BaO | 0-5 |
| ZnO | 0-4 |
| TiO₂ | 0-5 |
| ZrO₂ | 0-5 |
| TiO2 + ZrO₂ + SnO₂ | 2-6 |
| P₂O₅ | 0-8 |
| F | 0-1 |
| B₂O₃ | 0-2 |

Again preferably, the glass of a glass element to be used in the glass composite material according to the present disclosure can have the following composition (weight %):

| | |
|---|---|
| SiO₂ | 57-66 |
| Al₂O₃ | 18-23 |
| Li₂O | 3-5 |
| Na₂O + K₂O | 3-25 |
| MgO + CaO + SrO + BaO | 1-4 |
| ZnO | 0-4 |
| TiO₂ | 0-4 |
| ZrO₂ | 0-5 |
| TiO2 + ZrO₂ + SnO₂ | 2-6 |
| P₂O₅ | 0-7 |
| F | 0-1 |
| B₂O₃ | 0-2 |

Again preferably, the glass of a glass element to be used in the glass composite material according to the present disclosure can have the following composition (weight %):

| | |
|---|---|
| SiO₂ | 57-63 |
| Al₂O₃ | 18-22 |
| Li₂O | 3.5-5 |
| Na₂O + K₂O | 5-20 |
| MgO + CaO + SrO + BaO | 0-5 |
| ZnO | 0-3 |
| TiO₂ | 0-3 |
| ZrO₂ | 0-5 |
| TiO2 + ZrO₂ + SnO₂ | 2-5 |
| P₂O₅ | 0-5 |
| F | 0-1 |
| B₂O₃ | 0-2 |

Again, preferably, the glass of a glass element to be used in the glass composite material according to the present disclosure can have the following composition according to a soda-lime silicate glass (in weight %):

| | |
|---|---|
| SiO₂ | 40-81 |
| Al₂O₃ | 0-6 |
| B₂O₃ | 0-5 |
| Li₂O + Na₂O + K₂O | 5-30 |
| MgO + CaO + SrO + BaO + ZnO | 5-30 |
| TiO₂ + ZrO₂ | 0-7 |
| P₂O₅ | 0-2 |

Again preferably, the glass of a glass element to be used in the glass composite material according to the present disclosure can have the following composition (weight %):

| | |
|---|---|
| SiO₂ | 50-81 |
| Al₂O₃ | 0-5 |
| B₂O₃ | 0-5 |
| Li₂O + Na₂O + K₂O | 5-28 |
| MgO + CaO + SrO + BaO + ZnO | 5-25 |
| TiO₂ + ZrO₂ | 0-6 |
| P₂O₅ | 0-2 |

Again preferably, the glass of a glass element to be used in the glass composite material according to the present disclosure can have the following composition (weight %):

| | |
|---|---|
| SiO₂ | 50-76 |
| Al₂O₃ | 0-5 |
| B₂O₃ | 0-5 |
| Li₂O + Na₂O + K₂O | 5-25 |
| MgO + CaO + SrO + BaO + ZnO | 5-20 |
| TiO₂ + ZrO₂ | 0-5 |
| P₂O₅ | 0-2 |

Again preferably, the glass of a glass element to be used in the glass composite material according to the present disclosure can have the following composition according to a borosilicate glass (in weight %):

| | |
|---|---|
| SiO₂ | 60-85 |
| Al₂O₃ | 0-10 |
| B₂O₃ | 5-20 |
| Li₂O + Na₂O + K₂O | 2-16 |
| MgO + CaO + SrO + BaO + ZnO | 0-15 |
| TiO₂ + ZrO₂ | 0-5 |
| P₂O₅ | 0-2 |

Again preferably, the glass of a glass element to be used in the glass composite material according to the present disclosure can have the following composition (weight %):

| | |
|---|---|
| SiO₂ | 63-84 |
| Al₂O₃ | 0-8 |
| B₂O₃ | 5-18 |
| Li₂O + Na₂O + K₂O | 3-14 |
| MgO + CaO + SrO + BaO + ZnO | 0-12 |
| TiO₂ + ZrO₂ | 0-4 |
| P₂O₅ | 0-2 |

Again preferably, the glass of a glass element to be used in the glass composite material according to the present disclosure can have the following composition (weight %):

| | |
|---|---|
| SiO₂ | 63-83 |
| Al₂O₃ | 0-7 |
| B₂O₃ | 5-18 |
| Li₂O + Na₂O + K₂O | 4-14 |
| MgO + CaO + SrO + BaO + ZnO | 0-10 |
| TiO₂ + ZrO₂ | 0-3 |
| P₂O₅ | 0-2 |

Again preferably, the glass of a glass element to be used in the glass composite material according to the present disclosure can have the following composition according to an alkali-aluminum silicate glass (weight %):

| | |
|---|---|
| SiO₂ | 40-75 |
| Al₂O₃ | 10-30 |
| B₂O₃ | 0-20 |
| Li₂O + Na₂O + K₂O | 4-30 |
| MgO + CaO + SrO + BaO + ZnO | 0-15 |
| TiO₂ + ZrO₂ | 0-15 |
| P₂O₅ | 0-10 |

Again preferably, the glass of a glass element to be used in the glass composite material according to the present disclosure can have the following composition according to an alkaline aluminum silicate glass (weight %):

| | |
|---|---|
| SiO₂ | 50-70 |
| Al₂O₃ | 10-27 |
| B₂O₃ | 0-18 |
| Li₂O + Na₂O + K₂O | 5-28 |
| MgO + CaO + SrO + BaO + ZnO | 0-13 |
| TiO₂ + ZrO₂ | 0-13 |
| P₂O₅ | 0-9 |

Again preferably, the glass of a glass element to be used in the glass composite material according to the present disclosure can have the following composition (weight %):

| | |
|---|---|
| SiO₂ | 55-68 |
| Al₂O₃ | 10-27 |
| B₂O₃ | 0-15 |
| Li₂O + Na₂O + K₂O | 4-27 |
| MgO + CaO + SrO + BaO + ZnO | 0-12 |
| TiO₂ + ZrO₂ | 0-10 |
| P₂O₅ | 0-8 |

Again preferably, the glass of a glass element to be used in the glass composite material according to the present disclosure can have the following composition (weight %):

| | |
|---|---|
| SiO₂ | 50-75 |
| Al₂O₃ | 7-25 |
| B₂O₃ | 0-20 |
| Li₂O + Na₂O + K₂O | 0-4 |
| MgO + CaO + SrO + BaO + ZnO | 5-25 |
| TiO₂ + ZrO₂ | 0-10 |
| P₂O₅ | 0-5 |

Again preferably, the glass of a glass element to be used in the glass composite material according to the present disclosure can have the following composition (weight %):

| | |
|---|---|
| SiO₂ | 52-73 |
| Al₂O₃ | 7-23 |
| B₂O₃ | 0-18 |
| Li₂O + Na₂O + K₂O | 0-4 |
| MgO + CaO + SrO + BaO + ZnO | 5-23 |
| TiO₂ + ZrO₂ | 0-10 |
| P₂O₅ | 0-5 |

Again preferably, the glass of a glass element to be used in the glass composite material according to the present disclosure can have the following composition (weight %):

| | |
|---|---|
| SiO₂ | 53-71 |
| Al₂O₃ | 7-22 |
| B₂O₃ | 0-18 |
| Li₂O + Na₂O + K₂O | 0-4 |
| MgO + CaO + SrO + BaO + ZnO | 5-22 |
| TiO₂ + ZrO₂ | 0-8 |
| P₂O₅ | 0-5 |

It is understood that the respective glass components of the listed glass compositions must be 100% by weight in total. Nevertheless, the glasses to be used in the present disclosure can be modified, in particular the glasses described above. For example, the colour of the respective glass can be changed.

In advantageous embodiments, the glass elements are produced using particularly pure raw materials to minimize fluorescence under illumination with ultraviolet (UV) radiation and/or radiation in visible light. In particular, the use of raw materials with very low iron content has proven to be advantageous in this regard. The so prepared glasses thus advantageously contain particularly few impurities, especially little iron.

In another embodiment, the present disclosure solves the above objects with a device, in particular a device for use in biotechnological analysis methods comprising a base body made of the glass composite material according to the present disclosure, wherein the base body comprises one or more passageways, in particular a passageway or multiple passageways formed as channel or channels for liquids.

Advantageously, the first glass element can be formed as a bottom plate of the device and the second glass element as a cover plate of the device.

With regard to the configuration of the second glass element for realising the passageway in the base body, it is conceivable that the passageway or passageways are formed as recess or recesses in the second glass element. Preferably, the recesses can be shaped so that their geometry produces a particular flow behaviour of liquids flowing through the passageway or passageways.

In a further advantageous manner, the first glass element can be formed as a bottom plate of the device and the third glass element as a cover plate of the device. Here, the second glass element acts as an interposer (or spacer or intermediate piece) between the first and third glass element. In a particularly advantageous manner, in such embodiments, the second glass element can comprise one or more openings, wherein the opening or openings are formed in the second glass element such that the space formed by the opening or openings or the formed spaces in the device form the passageways. Alternatively or additionally, in further advantageous embodiments, the second glass element can comprise several parts, wherein the individual parts of the second glass element are formed such that the space or the spaces between the individual parts form the passageway or passageways.

In preferred embodiments, a first glass element formed as a bottom plate provides the device with stability, which facilitates the handling of the device, a second glass element designed as an interposer determines the geometry, in particular the height and width, of the passageway or passageways, and thereby the volume of the passageway or passageways, and the third glass element formed as a cover plate is selected in accordance with the analysis method in which the device is to be used, such that interference-free and high-resolution detection of the analysis signal is possible.

In an advantageous manner, the first glass element, in particular a first glass element formed as a bottom plate, is between 0.5 and 2.0 mm thick, in particular 0.5 mm 0.6 mm, 0.7 mm, 0.8 mm, 0.9 mm or 1, 0 mm thick, to give the device stability and facilitate handling of the device.

In a further advantageous manner, the second glass element, in particular a second glass element formed as an interposer, is a glass sheet having a thickness of between 0.05 and 0.3 mm, in particular between 0.1 and 0.175 mm. In a particularly advantageous manner, the second glass element, in particular a second glass element formed as an interposer, is a glass sheet having a thickness of 0.05 mm, 0.075 mm, 0.1 mm, 0.125 mm, 0.15 mm or 0.175 mm. As a result, advantageously the volume of passageway can be kept extremely low, so that the amounts of expensive and/or aggressive or toxic reagents to be used in the analysis method can be minimized.

In an advantageous manner, the third glass element, in particular a third glass element formed as a cover plate, is between 0.1 and 0.5 mm thick, in particular between 0.15 and 0.2 mm thick. In a particularly advantageous manner, the third glass element, in particular a third glass element formed as a cover plate, is 0.1 mm, 0.15 mm, 0.2 mm or 0.25 mm thick. As a result, the distance between an analysis signal to be detected within the passageway of the device and an optical amplifier or detector, for example microscope optics used in fluorescence microscopy, can also be kept small, so that interference-free and high-resolution detection of the analysis signal is possible.

In a particularly advantageous manner, in all of the described embodiments of the device, a plurality of the second silane coupling agents can be bonded to at least one surface of the passageway or the passageways. Alternatively or additionally, a plurality of the first silane coupling agents can be bonded to at least one surface of the passageway or the passageways. This allows for the immobilization of biomolecules being complementarily reactive to the first and/or second silane coupling agents on the respective surface of the passageway or passageways from a biological sample dissolved in a fluid, which is being guided through the passageway or passageways, such that, on the one hand, the composition of the sample and, on the other hand, the immobilized biomolecule itself can be analysed.

The passageway or passageways of the device have at least one inlet port and one outlet port through which biological samples dissolved in fluid can enter or can be introduced into the passageway and can exit or can be removed from the passageway, respectively. Due to the easy-to-implement precision shaping of the glass elements and the reproducible thickness of the coupling agent layer, the volume of a passageway in the device can be determined with high accuracy. In this way, the volume of a sample dissolved in liquid can be adapted to the volume of the passage so that, optionally, by complete filling of the passageway, not only the bottom surface of the passageway is contacted with sample, but also the side and ceiling surfaces. Likewise, the entire volume of the passageway can be easily flushed with washing solutions so that unfavourable impurities can be avoided.

Through the configuration of the second glass element, further advantageously, several microfluidic passageways can also lead through the device and thus allow for the parallel analysis of a plurality of samples, wherein the risk of cross-contamination is low.

Furthermore, the base body of the device can comprise fastening elements. For example, a first glass element formed as a bottom plate of the device can include elements for mounting the bottom plate in a laboratory machine for introducing samples and solutions into the device. The base body can further comprise attachment elements for analysis instruments or for in- or outflow conduits, which in some embodiments can also be in fluid communication with a passageway or multiple passageways of the device.

The device according to the present disclosure is suitable for use in biotechnological analysis methods in a particularly advantageous manner, in particular in methods, which require the use of expensive and/or only in the smallest amounts available, fluid-dissolved reagents. The reactions common in such methods can take place in the passageway or in the passageways of the devices according to the present disclosure, which can advantageously be formed as microfluidic channels and/or reaction chambers.

In various embodiments, the device is a microarray, a biochip or a flow chamber. In particular, a device according to the present disclosure can be used as a microfluidic flow chamber (microfluidic flow cell), for example, in methods of the Next Generation Sequencings (NGS, the latest DNA sequencing technologies).

Advantageously, oligonucleotide molecules, which are introduced into the passageway or passageways, can be immobilized on still reactive silane coupling agents extending into the passageway or passageways, in particular on amino silane, epoxy silane and/or NHS silane coupling agents.

In NGS methods, these oligonucleotides are usually linker sequences to which the nucleotide sequence to be sequenced is in turn bound by hybridization and is presented in the passageway of the device for the subsequent enzymatic polymerase reactions. An NGS method in a microfluidic passageway or channel, allows increased reaction efficiency since the temperatures necessary for the respective reaction steps are achieved particularly fast and without unfavourable delay in the small volumes.

In devices according to the present disclosure, which comprise a plurality of passageways, a plurality of different microfluidic methods can advantageously be carried out in the individual passageways in parallel, so that the device is used as a miniature laboratory or biochip.

In another embodiment, the present disclosure solves the aforementioned objects with a method for producing a glass composite material according to the present disclosure, comprising: providing a first glass element comprising a first surface to which a plurality of first silane coupling agents are bonded, as well as a second glass element which comprises a first surface to which a plurality of second silane coupling agents are bonded or comprising a first surface to which a plurality of second silane coupling agents are bonded and comprising a second surface to which a plurality of second silane coupling agents are bonded; and contacting the first surface of the first glass element with the first surface of the second glass element, so that the first and second silane coupling agents enter into covalent bonds and form a coupling agent layer between the first and second glass element, so that the first glass element is irreversibly connected to the second glass element.

In an advantageous manner, the method according to the present disclosure can also comprise providing a third glass element comprising a first surface to which a plurality of first silane coupling agents are bonded; and contacting the first surface of the third glass element with the second surface of the second glass element, so that the first and second silane coupling agents enter into covalent bonds and form a coupling agent layer between the second and third glass element, so that the second glass element is irreversibly connected to the third glass element.

In a further embodiment, the present disclosure solves the aforesaid objects with a method for producing a device according to the present disclosure comprising: providing a first glass element comprising a first surface to which a plurality of first silane coupling agents are bonded, as well as a second glass element which: comprises a first surface to which a plurality of second silane coupling agents are bonded; or comprises a first surface to which a plurality of second silane coupling agents are bonded and comprises a second surface to which a plurality of second silane coupling agents are bonded, and wherein the second glass element comprises one or more recesses or openings for forming the passageway or passageways; or wherein the second glass element comprises several parts, wherein the individual parts of the second glass element are formed, so that the space or the spaces between the individual parts form the passageway or passageways; and contacting the first surface of the first glass element with the first surface of the second glass element, so that the first and second silane coupling agents enter into covalent bonds and the interconnected glass elements form the base body of the device.

In an advantageous manner, the method according to the present disclosure can also comprise: providing of a third glass element comprising a first surface to which a plurality of first silane coupling agents are bonded; and contacting the first surface of the third glass element with the second surface of the second glass element, so that the first and second silane coupling agents enter into covalent bonds and the interconnected glass elements form the base body of the device.

Both in the described method for preparing the glass composite material according to the present disclosure as well as in the described method for producing the device according to the present disclosure, the contacting is carried out under conditions, which allow for the bonding reaction between the respective silane coupling agents to occur. Based on his/her common general knowledge, these conditions are readily accessible for those skilled in the art.

For example, in favourable embodiments, the contacting can comprise pressing the respective surfaces of the glass elements against each other. In a further advantageous manner, this pressing can be performed for a period of between 10 seconds and 12 hours, preferably between one minute and one hour, more preferably between 5 minutes and 30 minutes.

In a further advantageous manner, the contacting can be performed in a humid atmosphere, in particular in an atmosphere with a relative humidity of between 30 and 95%, preferably between 25 and 75%, more preferably between 50 and 75%. In a particularly advantageous manner, the contacting can be performed in a humid atmosphere with a relative humidity of 30, 35, 40, 45, 50, 55, 60, 65, 70, 75, 80, 85, 90, or 95%. In a particularly advantageous manner, the contacting can be performed at a temperature of between 10 and 50° C., in particular at a temperature of 15 to 35° C., in particular at a temperature of 20 to 30° C., in particular at 25° C.

In a surprisingly simple manner, the methods according to the present disclosure enable the production of high quantities of the glass composite materials and devices according to the present disclosure with particularly good manufacturing tolerance.

In another embodiment, the present disclosure solves the above objects through the use of the device according to the present disclosure for analysis of biological samples, preferably comprising oligonucleotides, bacterial artificial chromosome, peptides, proteins and glycans.

There are now several possibilities to realise and further develop the disclosure of the present disclosure advantageously.

### BRIEF DESCRIPTION OF THE DRAWINGS

FIG. 1 shows a schematic cross section through a glass composite according to the present disclosure comprising two glass elements.

FIG. 2 shows a device according to the present disclosure comprising two glass elements.

FIG. 3 shows a schematic perspective view of a device according to the present disclosure comprising three glass elements.

FIG. 4 shows a schematic cross section through a device according to the present disclosure as shown in FIG. 3 along A-A.

FIG. 5 shows a schematic cross section through a device according to the present disclosure as shown in FIG. 3 along B-B.

FIG. 6 shows a schematic perspective view of a second glass element (interposer) with openings according to the present disclosure.

### DETAILED DESCRIPTION

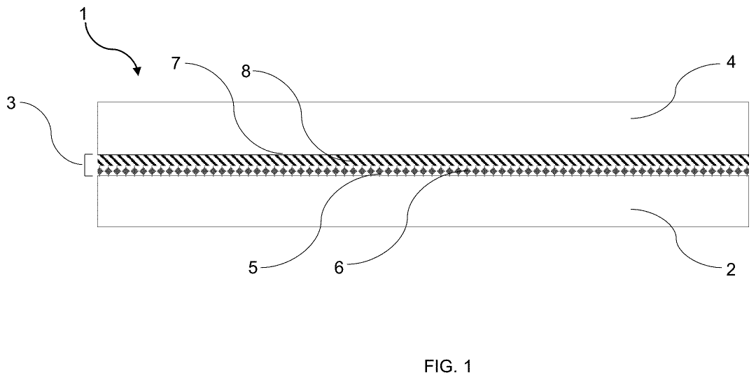

A glass composite material 1 according to the present disclosure is shown schematically in FIG. 1. The glass composite material consists of a first glass element 2, a coupling agent layer 3 and a second glass element 4. On the one hand, a plurality of first silane coupling agents 6 is covalently bonded to a first surface 5 of the first glass element 2. On the other hand, a plurality of second silane coupling agents 8 is covalently bonded to a first surface 7 of the second glass element 4. By complementary reactivity of the first silane coupling agents 6 and the second coupling agents 8, these enter into covalent bonds with each other and thus form the coupling agent layer 3, whereby the first glass element is irreversibly connected to the second glass element.

The glass composite material according to the present disclosure can, of course, comprise further glass elements, in particular a plurality of further glass layers, which are covalently and irreversibly connected to the first glass element 2 and/or the second glass element 4 and possibly also again with each other. The complementarily reactive silane coupling agents for connecting further glass elements to the first glass element 2 or the second glass element 4 of the glass composite material shown in FIG. 1 can again be the first silane coupling agents 6 and second silane coupling agents 8 or can comprise further complementarily reactive silane coupling agents.

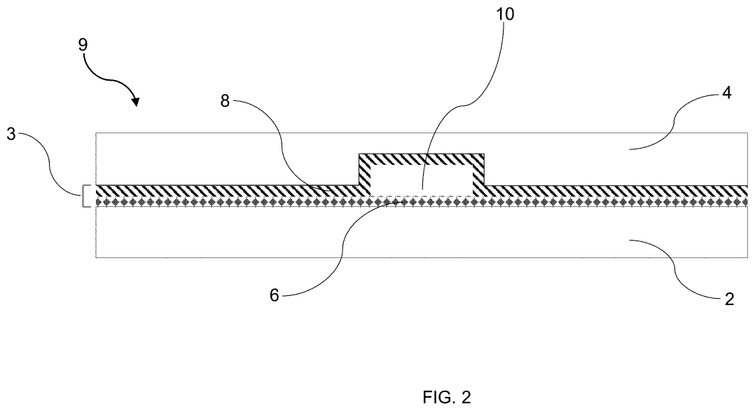

The glass composite material 1 according to the present disclosure can advantageously be used in a device according to the present disclosure, for example, in a device according to FIG. 2. In the device 9 shown schematically in FIG. 2, a one-piece second glass element 4 is shown that is covalently and irreversibly connected to the first glass element 2 via a coupling agent layer 3, wherein the passageway 10 of the device 9 is configured as a recess in the second glass element 4.

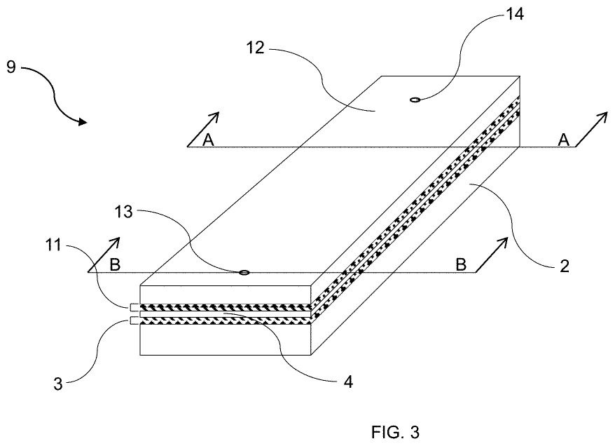

The device 9 according to the present disclosure shown in FIG. 3 comprises a second glass element 4 (interposer, originally coated at its first and second surfaces with a plurality of second silane coupling agents), which is covalently connected to a first glass element 2 designed as a bottom plate as well as to a third glass element 12 designed as a cover via two coupling agent layers 3, 11. In this embodiment, both the first and the third glass element was originally coated with a plurality of first silane coupling agents 6, so that both coupling agent layers 3, 11, are formed through a bonding reaction between the first and second silane coupling agents 6, 8. The passageway 10 of the device 9 is formed by the space in the opening (internally and not visible here) of the second glass element 4. The passageway is in fluid communication with both the inlet port 13 and the outlet port 14, so that samples dissolved in liquid and/or reagents can be introduced through the inlet port 13 for analysis in the passageway 10 and can be removed again via the outlet port 14.

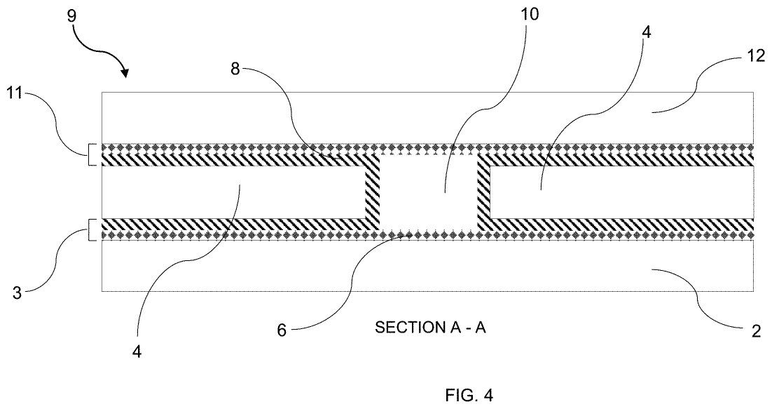

As can be seen from the cross section of FIG. 4 shown along A-A, the first glass element 2 and the third glass element 12 limit the space formed by the opening 14 in the second glass element 4, so that the passageway 10 of the device 9 shown in FIG. 3 is formed.

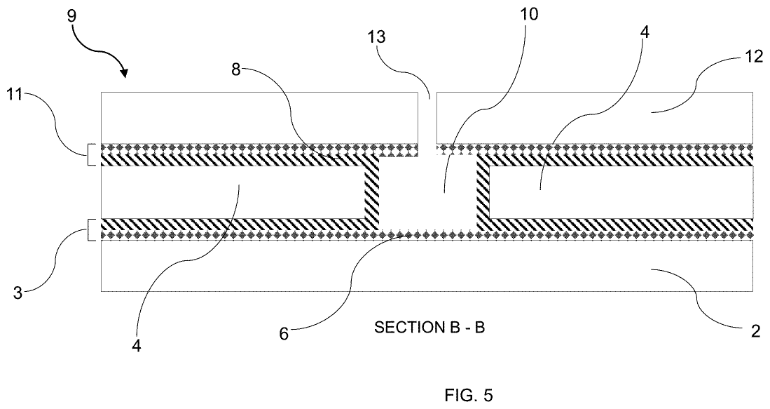

From the cross section along B-B shown in FIG. 5, it can be seen that the inlet port 13 is in fluid communication with the opening 14 of the second glass element 4 (interposer), and thus with the passageway 10 of the device 9 shown in FIG. 3, so that in a liquid dissolved samples and/or reagents can be introduced through the inlet port 13 for analysis in the passageway 10.

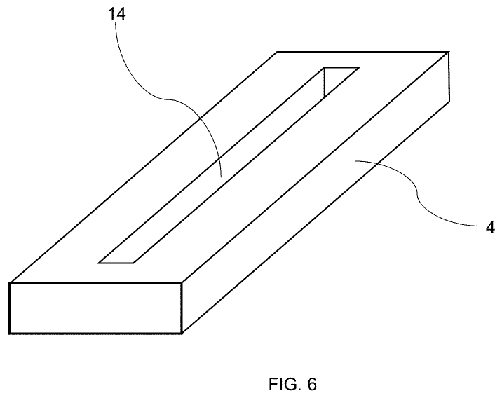

FIG. 6 shows the second glass element 4 designed as an interposer with opening 14 in a schematic perspective view.

With regard to further advantageous embodiments of the device according to the present disclosure, reference is made to the general part of the description and to the appended claims in order to avoid repetition.

Finally, it should be expressly noted that the examples of embodiments of the device according to the present disclosure described above serve merely to illustrate the claimed teaching, but do not restrict it to the examples of embodiments.

What is claimed is:

1. A glass composite, comprising:
a first glass element having a first surface;
a second glass element having a first surface; and
a first layer having a first silane coupling agent and a second silane coupling agent,
wherein the first layer has covalent bonds between the first and second silane coupling agents,
wherein the first silane coupling agent is covalently bonded to the first surface of the first glass element,
wherein the second silane coupling agent is covalently bonded to the first surface of the second glass element, and
wherein the first glass element is irreversibly connected to the second glass element by the first layer.

2. The glass composite of claim 1, further comprising:
a third glass element having a first surface; and
a second layer having the first silane coupling agent and the second silane coupling agent,
wherein the second layer has covalent bonds between the first and second silane coupling agents,
wherein the second glass element is irreversibly connected to the third glass element by the second layer,
wherein the first silane coupling agent is covalently bonded to a second surface of the second glass element, and
wherein the second silane coupling agent is covalently bonded to the first surface of the third glass element.

3. The glass composite of claim 1, further comprising:
a third glass element having a first surface; and
a second layer having the first silane coupling agent and the second silane coupling agent,
wherein the second layer has covalent bonds between the first and second silane coupling agents,
wherein the second glass element is irreversibly connected to the third glass element by the second layer,
wherein the second silane coupling agent is covalently bonded to a second surface of the second glass element, and
wherein the first silane coupling agent is covalently bonded to the first surface of the third glass element.

4. The glass composite of claim 1, wherein the first silane coupling agent is selected from the group consisting of: a reactive epoxy, an aldehyde group, a polymer group, and combinations thereof, and wherein the second silane coupling agent is a reactive amino group.

5. The glass composite of claim 1, wherein the second silane coupling agent is selected from the group consisting of: a reactive epoxy, an aldehyde group, a polymer group, and combinations thereof, and wherein the first silane coupling agent is a reactive amino group.

6. The glass composite of claim 1, wherein the first silane coupling agent comprises one or more reactive epoxy groups and the second silane coupling agent is a reactive thiol group.

7. The glass composite of claim 1, wherein the second silane coupling agent comprises one or more reactive epoxy groups and wherein the first silane coupling agent is a reactive thiol group.

8. The glass composite of claim 1, wherein the first and second glass elements are selected from the group consisting of: a soda-lime glass element, a borosilicate glass element, a quartz glass element, an alkaline alumino borosilicate glass element, and combinations thereof.

9. The glass composite of claim 1, further comprising a passageway configured as a channel for liquids such that the glass composite is configured for use in a biotechnological analysis method.

10. The glass composite of claim 9, wherein the passageway is a recess in the second glass element.

11. The glass composite of claim 9, wherein the second glass element comprises one or more openings that form the passageway.

12. The glass composite of claim 9, wherein the second glass element comprises at least two parts with a space therebetween that forms the passageway.

13. The glass composite of claim 9, wherein the passageway has at least one surface and wherein the second silane coupling agent is bonded to the at least one surface of the passageway.

14. The glass composite of claim 9, wherein the passageway has at least one surface and wherein the first silane coupling agent is bonded to the at least one surface of the passageway.

15. The glass composite of claim 9, wherein the second glass element is a glass sheet having a thickness from 0.05 to 0.3 mm.

16. The glass composite of claim 9, wherein further comprising a fastening element on the first and/or second glass element, the fastening element being configured to mount the glass composite in a laboratory machine that conducts the biotechnological analysis method.

17. The glass composite of claim 9, wherein the glass composite is configured as a device selected from the group consisting of: a microarray, a biochip, and a flow chamber.

18. A method for producing a glass composite, comprising:
bonding a first surface of a first glass element to a first silane coupling agent;
bonding a first surface of a second glass element to a second silane coupling agent; and
contacting the first and second glass elements so that the first and second silane coupling agents covalently bond to one another and irreversibly connect the first and second glass elements.

19. The method of claim 18, further comprising:
bonding a second surface of the second glass element to a second silane coupling agent;
bonding a first surface of a third glass element to a first silane coupling agent; and
contacting the second and third glass elements so that the first and second silane coupling agents covalently bond to one another and irreversibly connect the second and third glass elements to define the glass composite.

20. The method of claim 18, further comprising forming a passageway in the glass composite such that the glass composite is configured for use in a biotechnological analysis method.

21. The method of claim 20, wherein the step of forming the passageway comprises:
defining one or more recesses or openings in the second glass element that form the passageway; and/or
using a plurality of elements to define the second glass element and positioning the plurality of elements with a space therebetween to form the passageway.
